(19) United States
(12) Patent Application Publication
(10) Pub. No.: US 2026/0165865 A1
(43) Pub. Date: Jun. 18, 2026

(54) KNEE ORTHOSIS FOR A TRANS-TIBIAL AMPUTEE HAVING LEG PROSTHESIS

(71) Applicant: OSSKIN ORTHO INC., Montréal (CA)

(72) Inventors: Pierre COUTU, Montreal (CA); Pascale BRASSARD, Montreal (CA); Victor GRENIER, Montréal (CA); Farah OUALI, Montréal (CA)

(21) Appl. No.: 19/307,608

(22) Filed: Aug. 22, 2025

Related U.S. Application Data
(60) Provisional application No. 63/735,000, filed on Dec. 17, 2024.

(51) Int. Cl. A61F 5/01; B33Y 10/00; B33Y 80/00

(52) U.S. Cl. CPC A61F 5/0123; B33Y 10/00; B33Y 80/00; A61F 2005/0139; A61F 2005/0146

(57) ABSTRACT

A knee orthosis assembly for a trans-tibial amputee having a leg prosthesis includes a socket for receiving a stump of the trans-tibial amputee, the socket having an open end receiving the stump, the open end including a socket hinge element integrated with the open end. A femoral cuff is configured for engaging an upper leg portion of the trans-tibial amputee, the femoral cuff having a femoral transverse member and a femoral longitudinal member protruding from the femoral transverse member. The femoral longitudinal member includes a femoral hinge element integrated with the femoral longitudinal member, the femoral hinge element engaging with the socket hinge element to define a hinge of the knee orthosis assembly.

### CROSS-REFERENCE TO RELATED APPLICATIONS

The present application claims priority on U.S. patent application Ser. No. 63/735,000 filed Dec. 17, 2024, the entire content of which is incorporated herein by reference.

### TECHNICAL FIELD

The present disclosure relates generally to knee orthoses and, more particularly, to knee orthoses adapted for use by trans-tibial amputees.

### BACKGROUND

Trans-tibial amputees have their knee joint preserved but often have pathological biomechanics as the residual limb and prosthesis do not offer the same stability/control as the native limb. This may result in knee ligament deficiencies, which require stabilization and/or femoral axis correction (varus/valgus). While knee orthoses may be used to provide such stabilization, existing knee orthoses are typically not well suited to bracing the knees of trans-tibial amputees. Movement between the knee orthosis and the leg, or between the knee orthosis and the socket of a lower leg prosthesis, is undesirable as it can result in unwanted forces and/or migration between the wearer's leg and the knee orthosis. In addition, existing trans-tibial knee orthoses are lacking in modularity and adjustability. For trans-tibial amputees, the limited amount of soft tissue available to compensate for the relative position of the knee orthosis and the limb also causes greater challenges in ensuring that the knee orthosis accurately tracks the natural motion of the leg and knee. Improvements are therefore sought.

### SUMMARY

According to the present disclosure, there is provided a knee orthosis assembly for a trans-tibial amputee having a leg prosthesis, comprising: a socket for receiving a stump of the trans-tibial amputee, the socket having an open end receiving the stump, the open end including a socket hinge element integrated with the open end; and a femoral cuff configured for engaging an upper leg portion of the trans-tibial amputee, the femoral cuff having a femoral transverse member and a femoral longitudinal member protruding from the femoral transverse member, the femoral longitudinal member including a femoral hinge element integrated with the femoral longitudinal member, the femoral hinge element engaging with the socket hinge element to define a hinge of the knee orthosis assembly.

In certain embodiments, the knee orthosis assembly as defined above and described herein includes one or more of the following features, in whole or in part, and in any combination.

In certain embodiments, the socket hinge element directly engages the femoral hinge element.

In certain embodiments, the socket hinge element includes a first aperture and the femoral hinge element includes a second aperture aligned with the first aperture and rotatably secured to one another by a fastener inserted through the first and second apertures.

In certain embodiments, one of the socket hinge element and the femoral hinge element includes slots defined therethrough, and the other of the socket hinge element and the femoral hinge element includes apertures defined therethrough, the slots and apertures aligned and rotatably secured to one another by fasteners inserted therethrough.

In certain embodiments, an additional hinge element interfaces the socket hinge element to the femoral hinge element.

In certain embodiments, the socket hinge element and the femoral hinge element each include holes and toothed portions, the toothed portions engaging one another to allow the femoral cuff to pivot with respect to the socket, and wherein the additional hinge element includes support arms engaging the holes in the socket hinge element and the femoral hinge element to secure the socket to the femoral cuff assembly.

In certain embodiments, the socket hinge element and the femoral hinge element each include holes and the additional hinge element includes 4-bars links engaging the holes in the socket hinge element and the femoral hinge element to pivotally secure the socket to the femoral cuff.

In certain embodiments, the socket hinge element and the femoral hinge element each include holes and the additional hinge element includes a disk-shaped body having slots and holes disposed therethrough, the slots in the disk-shaped body engaging the holes in one of the socket hinge element and the femoral hinge element, the holes in the disk-shaped body engaging the holes in the other of the socket hinge element and the femoral hinge element to pivotally secure the socket to the knee orthosis assembly.

In certain embodiments, the additional hinge element is detachably secured to the socket hinge element and rotatably secured to the femoral hinge element.

In certain embodiments, an inner surface of the femoral cuff engages an outer surface of the socket adjacent the hinge.

In certain embodiments, an outer surface of femoral cuff engages an inner surface of the socket adjacent the hinge.

In certain embodiments, the socket hinge element includes a first aperture and the femoral hinge element includes a second aperture aligned with the first aperture and rotatably secured to one another by a fastener inserted through the first and second apertures.

In certain embodiments, one of the socket hinge element and the femoral hinge element includes slots defined therethrough and the other of the socket hinge element and the femoral hinge element includes apertures defined therethrough, the slots and apertures aligned and rotatably secured to one another by fasteners inserted therethrough.

In certain embodiments, the socket hinge element and the femoral hinge element each include holes and toothed portions, the toothed portions engaging one another to allow the femoral cuff to pivot with respect to the socket.

In certain embodiments, 4-bar links engage holes in the socket hinge element and the femoral hinge element to pivotally secure the socket to the femoral cuff.

In certain embodiments, the assembly includes a disk-shaped body having slots and holes disposed therethrough, the slots in the disk-shaped body engaging holes in one of the socket hinge element and the femoral hinge element, the holes in the disk-shaped body engaging holes in the other of the socket hinge element and the femoral hinge element to pivotally secure the socket to the femoral cuff.

In certain embodiments, an additional hinge element is detachably secured to the socket hinge element and rotatably secured to the femoral hinge element.

There is also provided a method of manufacturing a socket for a leg prosthesis of a trans-tibial amputee, the socket engageable with a knee orthosis, comprising: scanning a leg of the trans-tibial amputee to obtain information pertaining to anatomy of the leg; modelling the socket based on the anatomy of the leg, including modelling a socket hinge element integrated to the socket and engageable with a corresponding hinge element of the knee orthosis; and 3D printing the socket with the socket hinge element integrally formed therewith.

The method can also include modeling the socket hinge element to be directly engageable with corresponding hinge element of the knee orthosis.

There is further provided a knee orthosis assembly for a patient, comprising: a femoral cuff configured for engaging an upper leg portion of the patient, the femoral cuff having a femoral transverse member and a femoral longitudinal member protruding from the femoral transverse member, the femoral longitudinal member including a femoral hinge element integrated with the femoral longitudinal member; and an ankle-foot orthosis having a shank cuff receiving a lower leg portion of the patient, the shank cuff including an ankle-foot orthosis hinge element configured for engaging with the femoral hinge element, a shank coupled to the shank cuff and engaging an ankle of the patient, and a foot plate coupled to the shank and supporting a foot of the patient.

Many further features and combinations thereof concerning the present improvements will appear to those skilled in the art following a reading of the instant disclosure.

### BRIEF DESCRIPTION OF THE DRAWINGS

FIG. 1A-1C are three dimensional, front and cross-section views, respectively, of a knee orthosis assembly for a trans-tibial amputee in accordance with one implementation of the present technology;

FIG. 2 is an exploded, three-dimensional view of the knee orthosis assembly of FIGS. 1A-1C;

FIGS. 3A-3D are cross-section views of different implementations of the knee orthosis assembly of FIGS. 1A-1C;

FIG. 4 is an exploded, three-dimensional view of a knee orthosis assembly for a trans-tibial amputee in accordance with another implementation of the present technology;

FIG. 5 is a three-dimensional view of a socket for the knee orthosis assembly of FIG. 4;

FIGS. 6A and 6B are exploded three dimensional and enhanced and exploded three dimensional views, respectively, of a knee orthosis assembly for a trans-tibial amputee in accordance with yet another implementation of the present technology;

FIG. 7 is a three-dimensional view of a knee orthosis assembly for a trans-tibial amputee in accordance with yet another implementation of the present technology;

FIG. 8 is an exploded, three-dimensional view of a socket and an additional hinge element of a knee orthosis assembly for a trans-tibial amputee, in accordance with yet another implementation of the present technology;

FIG. 9 is an exploded, three-dimensional view of a socket and an additional hinge element of a knee orthosis assembly for a trans-tibial amputee, in accordance with yet another implementation of the present technology;

FIG. 10 is a three-dimensional view of a socket and an additional hinge element of a knee orthosis assembly for a trans-tibial amputee, in accordance with yet another implementation of the present technology;

FIG. 11 is a partially exploded, three-dimensional view of a socket and an additional hinge element of a knee orthosis assembly for a trans-tibial amputee, in accordance with yet another implementation of the present technology;

FIG. 12 is a partially exploded, three-dimensional view of a socket and an additional hinge element of a knee orthosis assembly for a trans-tibial amputee in accordance with yet another implementation of the present technology;

FIG. 13A is a front view of a knee orthosis assembly for a trans-tibial amputee in accordance with another implementation of the present technology;

FIG. 13B is a three-dimensional view of the knee orthosis assembly of FIG. 13A; and

FIG. 14 is a three-dimensional view of an ankle foot orthosis assembly in accordance with another implementation of the present technology.

### DETAILED DESCRIPTION

Knee orthoses are externally applied devices used to influence the structural and functional characteristics of the neuromuscular and skeletal systems. A knee orthosis is a brace that extends above and below the knee joint and is generally worn to support or align the knee during flexion thereof. In the case of conditions affecting the ligaments or cartilage of the knee, a knee orthosis can provide stabilization and/or varus/valgus correction and unloading relief to the knee by replacing or assisting the function of these injured or damaged ligaments or cartilage of the knee.

Stabilizing a transtibial amputee's knee using a knee orthosis (which may also be referred to herein as a “knee brace” or simply “brace”) offers a number of advantages, as it is easily removable and can be worn whenever the patient or clinician feels its necessary (e.g., for higher intensity activities). The residual limb (or stump) and socket can have varying shapes and sizes from one patient to another. Furthermore, there is no soft tissue on the tibia to compensate for discrepancies in socket vs. knee orthosis surface fit. Therefore, a custom, scan-based solution is preferred to a prefab knee orthosis with a typically cylindrical rigid cuff.

It is desirable for the knee orthosis to properly track a natural motion of the leg and knee (with a plurality of DOFs, screw-home mechanism, etc.) during flexion. If the leg and knee orthosis do not move together, there may be undesirable forces and slippage (i.e., migration) between the two.

This effect is worsened with trans-tibial amputees, as less soft tissue (skin, muscles, fat) exists to compensate for the relative position of the knee orthosis relative to the limb. As such, conventional knee orthoses are not well suited to bracing these patients. Put differently, gravity combined with repeated movements of the leg of the transtibial amputee may cause the knee orthosis to become misaligned relative to a leg prosthesis of the amputee. The relative migration of the knee orthosis may prevent the latter from properly assisting a knee flexion of the amputee.

In addition, trans-tibial knee orthoses may be customized for an individual patient based on their specific anatomy, and an orthosis produced having a femoral portion and a hinge mechanism for connecting the femoral portion to the socket. However, such systems typically require fixed or non-removable attachment of the hinge mechanism to the socket, thereby limiting further modifications or adjustments. The present knee orthosis seeks to address at least some of these issues.

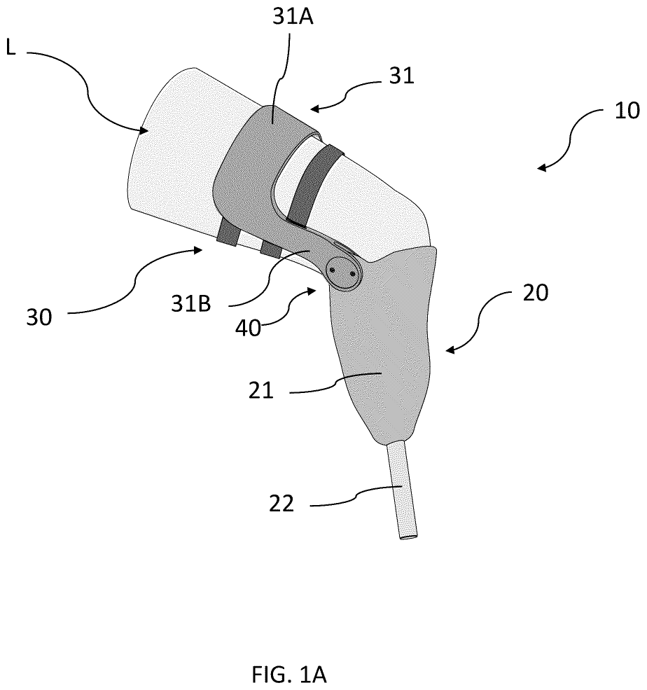

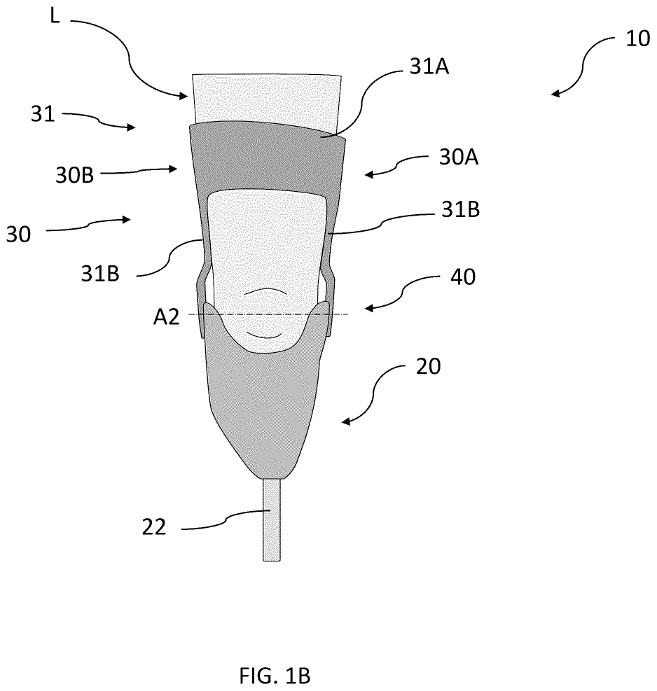

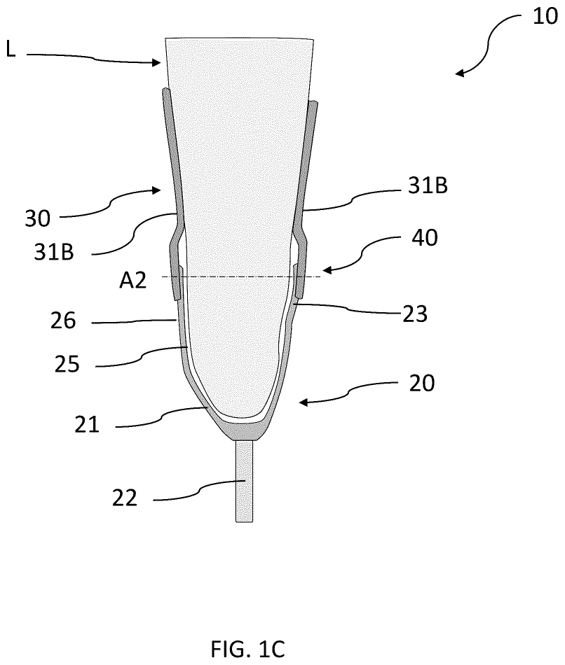

Referring to FIGS. 1A-1C, a knee orthosis assembly 10 of a leg prosthesis 20 for a trans-tibial amputee with leg L and a knee orthosis 30 is shown that may at least partially alleviate the aforementioned drawbacks. The leg prosthesis is shown at 20 and the knee orthosis is shown at 30. The leg prosthesis 20 includes a socket 21 for receiving a stump of the trans-tibial amputee and a leg member 22 that extends from the socket 21 towards a foot (not shown). The knee orthosis 30 is used to help stabilize the socket 21 of the leg prosthesis 20. The knee orthosis 30 may be custom fitted using a 3D scan of the leg and prosthesis. As will be discussed in further detail below, according to the present disclosure, a hinge element 40 for allowing the knee orthosis 30 to pivot or rotate relative to socket 21 is at least partially integrated to both the knee orthosis 30 and the socket 21. In some embodiments, the tibial portion of the hinge 40 is directly embedded into the socket 21. In other embodiments, an intermediary hinge component is provided (see additional hinge element 50 below) to act as an interface and/or connective element between the hinge elements in the socket 21 and knee orthosis 30. The assembly of the tibial hinge element may thus be permanent or removable. The features disclosed herein of the various embodiments of a hinge element 40, whether as an additional component or as integrated with the socket 21 and/or knee orthosis 30, are understood to be interchangeable, where applicable.

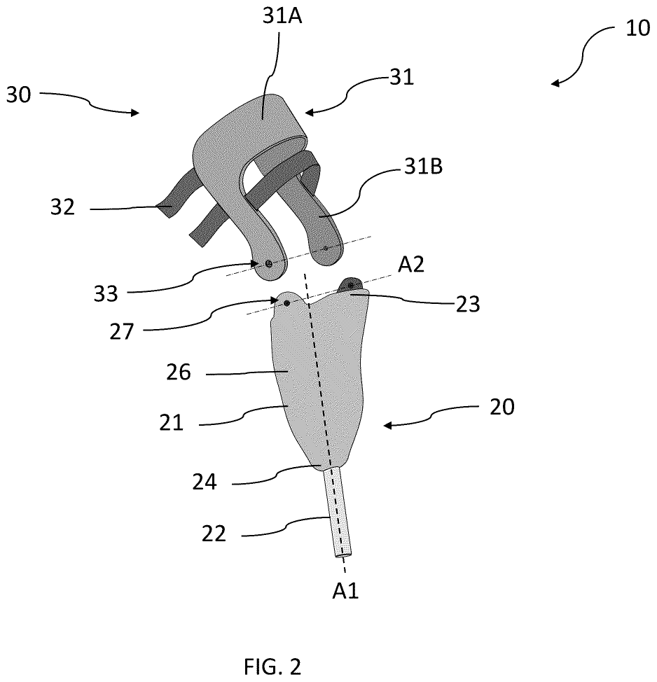

Referring additionally to FIG. 2, the socket 21 extends along a socket axis A1 from an open end 23 to a closed end 24. The wearer inserts an end of his or her leg into the socket 21 via the open end 23 until the end abuts the closed end 24. The socket axis A1 is therefore substantially parallel to the tibial bone of the wearer. In the context of the present disclosure, the expression “substantially” as in “substantially parallel” implies slight deviations from parallel due to an anatomy of the wearer, movements, and so on. The shape of the socket 21 is selected to substantially match that of the stump of the wearer. The socket 21 has an inner surface 25 facing an internal volume that receives the stump and an outer surface 26 opposed to the inner surface 25. The socket further includes socket hinge elements 27 towards the open end 23 of the socket 21, as will be discussed in further detail below.

The knee orthosis 30 includes a femoral cuff 31. The femoral cuff 31 extends partially around a thigh of the wearer and is securable to the thigh via any suitable means, such as straps 32. In other words, the femoral cuff 31 is configured for engaging an upper leg portion of the trans-tibial amputee. The femoral cuff 31 may have a curved shape configured to mate with a thigh of the wearer. The femoral cuff 31 is hingedly or pivotally connected to the socket 21 via a hinge or pivot 40 that is at least partially integrated with both the knee orthosis 30 and socket 21, as will be discussed in further detail below. The hinge 40 allows rotation of the tibial cuff 31 relative to the socket 21 about a pivot axis A2, which may be substantially coaxial with a pivot axis of the knee of the wearer.

The femoral cuff 31 may be manufactured using injection molding techniques and be manufactured in different sizes and configurations. In some implementations, the femoral cuff 31 is made using additive manufacturing techniques and based on a model of the wearer's knee obtained based on a scan and/or measurements of the wearer's knee. Such an implementation may provide for a custom fit of the femoral cuff 31 and may improve wearer's comfort.

As illustrated, the knee orthosis 30 has a medial side 30A and a lateral side 30B (see FIG. 1B). The femoral cuff 31 extends from the medial side 30A to the lateral side 30B of the knee orthosis 30. Put differently, the medial side 30A of the knee orthosis 30 and one side of the hinge 40 is located adjacent to a medial side of a knee of the wearer, and the lateral side 30B of the knee orthosis 30 and the other portion of the hinge 40 is located adjacent a lateral side of the knee. The knee orthosis 30, in particular the femoral cuff 31, defines a curved shape configured to substantially conform to a front section of the leg of the wearer. In an alternate embodiment, at least a portion of the femoral cuff 31 may be shaped to mate with a posterior side of the leg of the wearer.

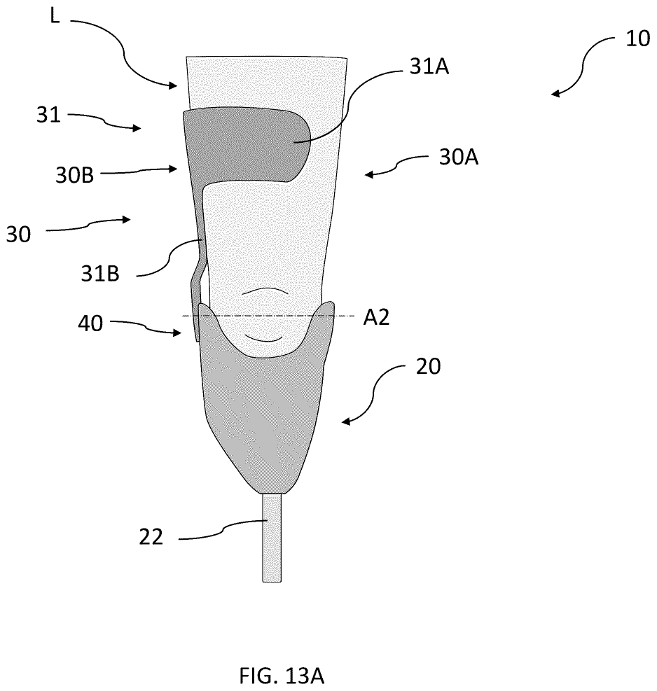

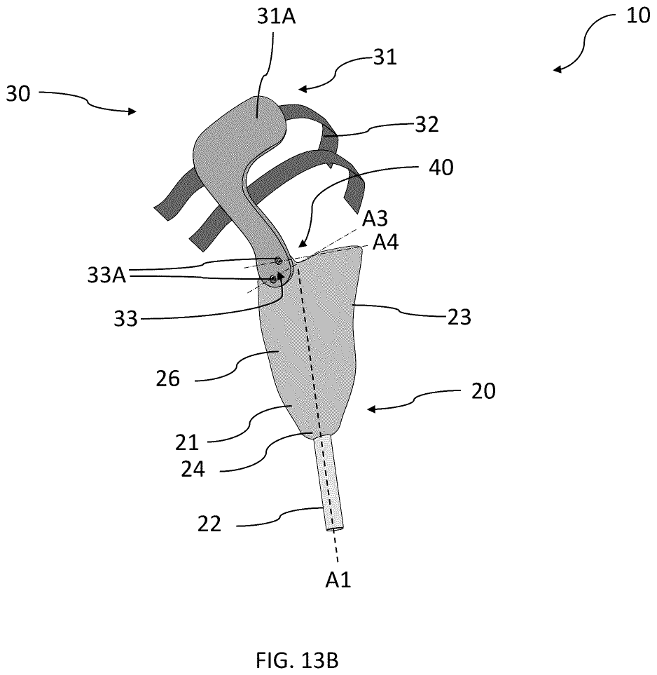

More particularly, the femoral cuff 31 has a femoral transverse member 31A wrapping around a portion of the thigh of the wearer and around a longitudinal axis of a femoral bone of the wearer and has one or two femoral longitudinal members 31B protruding from the femoral transverse member 31A towards the knee of the wearer and substantially along the longitudinal axis of the femoral bone. The knee orthoses 30 shown in FIGS. 1A-12 include two femoral longitudinal members 31B, while the knee orthosis 30 shown in FIGS. 13A-13B includes a single femoral longitudinal member 31B, as discussed in further detail below. Each of the femoral longitudinal members 31B includes a femoral hinge element 33 towards a lower end thereof. The femoral hinge elements 33 engage and cooperate with the socket hinge elements 27, with or without an intermediary component (as discussed in further detail below), to form the hinge 40, also referred to as a hinge element 40. As such, each of the two femoral longitudinal members 31B is pivotally connected to the open end 23 of the socket 21, with the hinge 40 at least partially integrated with the socket 21. In the shown case, the outer surface 26 of the socket 20 engages an inner surface of the femoral cuff 31. The opposite arrangement is contemplated, where the inner surface 25 of the socket 20 engages an outer surface of the femoral cuff 31.

An exemplary embodiment of the hinge 40 is shown in FIG. 2. In this embodiment, each of the femoral hinge elements 33 includes a single aperture formed through a respective femoral longitudinal member 31B. Likewise, each of the socket hinge elements 27 includes a single aperture formed through opposite ends of the open end 23 of the socket 21. Respective apertures through the femoral longitudinal member 31B and open end 23 of the socket 21 (i.e., a first aperture through a socket hinge element 27 and a second aperture through a femoral hinge element 33) are aligned along the pivot axis A2 and fastened via fasteners (not shown), for instance screws or pins. The knee orthosis 30 is thus pivotable with respect to the socket 21 about the pivot axis A2. In other cases, each of the socket 21 and the knee orthosis 30 include other numbers of apertures, for instance two apertures on either side of the hinge 40 (as shown in FIG. 1). Other configurations for the hinge 40 are contemplated, as discussed in further detail below.

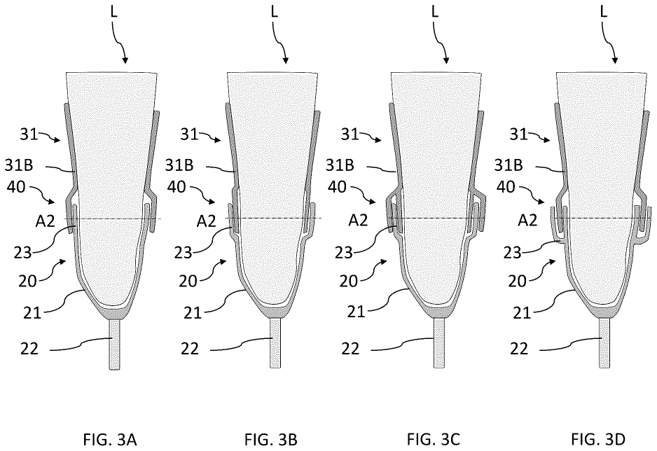

Referring now to FIGS. 3A-3D, various arrangements at the hinge 40 are contemplated. In the embodiment shown in FIG. 3A, the longitudinal members 31B of the femoral cuff 31 are disposed outwardly or externally of the socket 21 with respect to the pivot axis A2. In the embodiment shown in FIG. 3B, the longitudinal members 31B of the femoral cuff 31 are disposed inwardly or internally of the socket 21 with respect to the pivot axis A2. In the embodiment shown in FIG. 3C, the lower ends of the longitudinal members 31B have fork-like members that receives the socket 21 in cavities defined between the fork-like members. In the embodiment shown in FIG. 3D, the socket 21, towards the open end 23, has fork-like members on opposing sides thereof, the fork-like members receiving the longitudinal members 31B in cavities defined therein. Other arrangements are also contemplated.

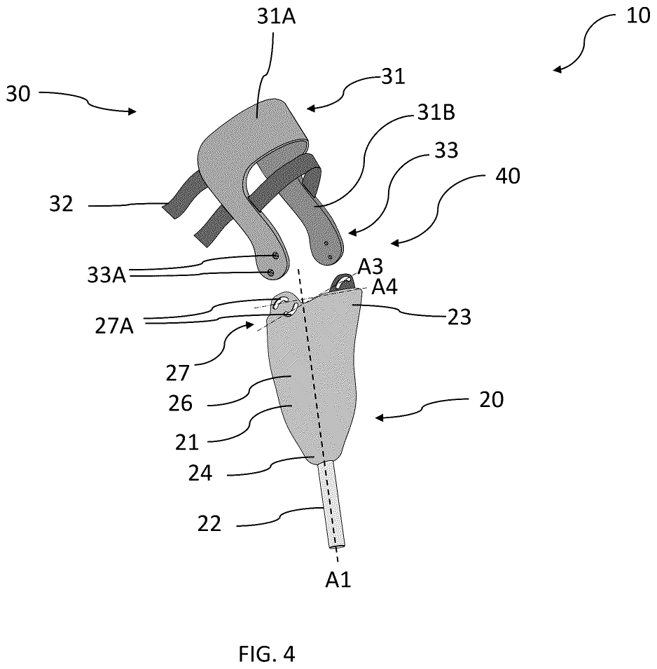

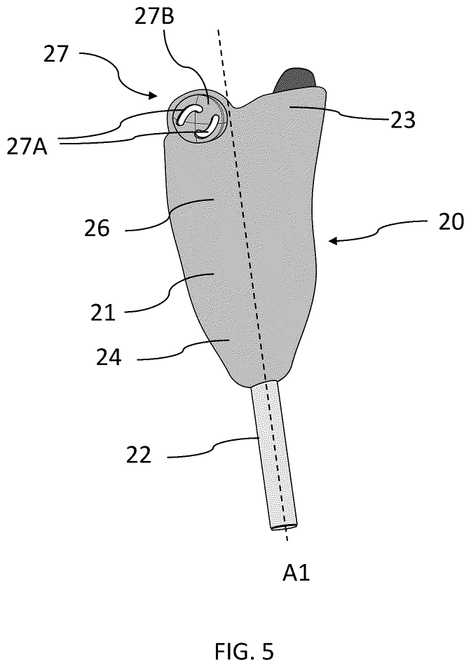

Referring now to FIGS. 4 and 5, another embodiment of a hinge 40 for the assembly 10 is shown. In this embodiment, each femoral hinge element 33 includes a pair of holes 33A extending therethrough, and each socket hinge element 27 includes a pair of guide slots 27A disposed therethrough. In another possible embodiment, however, the apertures 33A are disposed in the socket hinge element 27 and the guide slots 27A are disposed in the femoral hinge element 33. Accordingly, one of the socket hinge element 27 and the femoral hinge element 33 includes the slots 27A defined therethrough and the other of the socket hinge element 27 and the femoral hinge element 33 includes the apertures 33A defined therethrough. Additionally, it is also possible for each of the socket hinge element 27 and the femoral hinge element 33 to have a combination of slots 27A and apertures 33. Regardless of the configuration, the holes 33A and guide slots 27A are adapted to be aligned and receive pins (not shown) therein, the pins rotatably fastening the socket 21 to the femoral cuff 31. The guide slots 27A are thus adapted for guiding the pivot of the femoral cuff 31 relative to the socket 21 during flexion of the knee orthosis 30. The guide slots 27A are defined by tracking projections about a flexion axis defined by guide slot axes A3, A4 throughout a full flexion range of the knee orthosis 30. More particularly, the geometry of the guide slots 27A is chosen such that the relative movement between the guide slots 27A and the pins generate a predetermined movement controlling rotation and translation in various degrees of freedom about a virtual flexion axis, at each of a plurality of points through the range of travel, as the knee orthosis 30 moves through its full flexion range, such as to cause a desired movement of the knee. The area of the socket 21 surrounding the guide slots 27A may be flush with the outer surface 26 (as shown in FIG. 4) or be formed as part of a raised portion 27B protruding from the outer surface 26 (as shown in FIG. 5). Other configurations are contemplated, for instance the opposite arrangement whereby the socket 21 includes the holes and the knee orthosis 30 includes the guide slots.

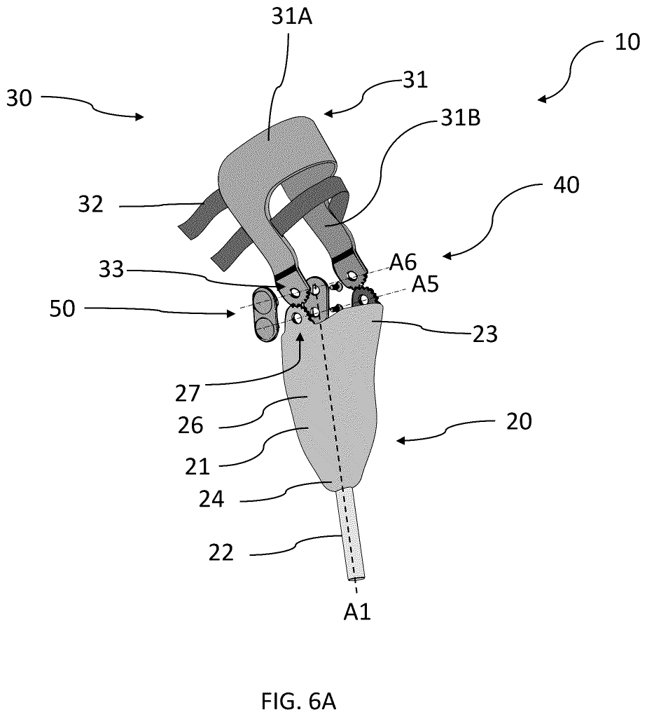

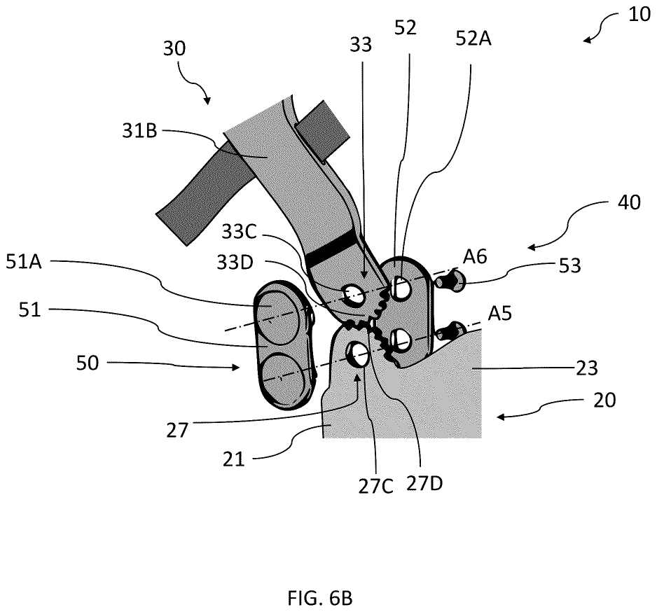

Referring now to FIGS. 6A-6B, another embodiment of a hinge 40 for the assembly 10 is shown, in which an additional hinge element 50 is provided for securing the knee orthosis 30 to the socket 21. In this embodiment, the hinge 40 is a polycentric hinge. The femoral hinge element 33 includes a hole 33C and a toothed portion 33D towards a lower end of each longitudinal member 31B. The socket hinge element 27 includes, towards the open end 23, a pair of holes 27C and a pair of toothed portion 27D on opposite ends of an upper extremity of the open end 23. The toothed portions 27D, 33D are configured to engage one another to allow the knee orthosis 30 to pivot with respect to the socket 21, with the movement guided by the additional hinge element 50. In particular, the additional hinge element in the embodiment of FIGS. 6A-6B includes a first support arm 51 having a pair of barrels nuts 51A, a second support arm 52 having a pair of holes 52A, and fasteners 53 (e.g., screws) on each side of the brace (medial and lateral). The support arms 51, 52 are positioned on opposing sides of a respective pair of a socket hinge element 27 and femoral hinge element 33 such that a given barrel nut 51A and hole 52A are aligned, along one of axes A5 and A6, with the one of the holes 33C, 27C, with a fastener 53 extending through each to secure the support arms 51, 52 together, thereby pivotally securing the knee orthosis 30 to the socket 21. Stated differently, the pivoting motion of the knee orthosis 30 relative to the socket 21 occurs due to relative rotation of the toothed portions 33D, 27D, and is delimited by the range of motion provided by the hinge mechanism 50. For clarity, FIG. 6B only shows the hinge 40 of the lateral side 30B. It is understood, however, that an equivalent configuration is provided on the medial side 30A.

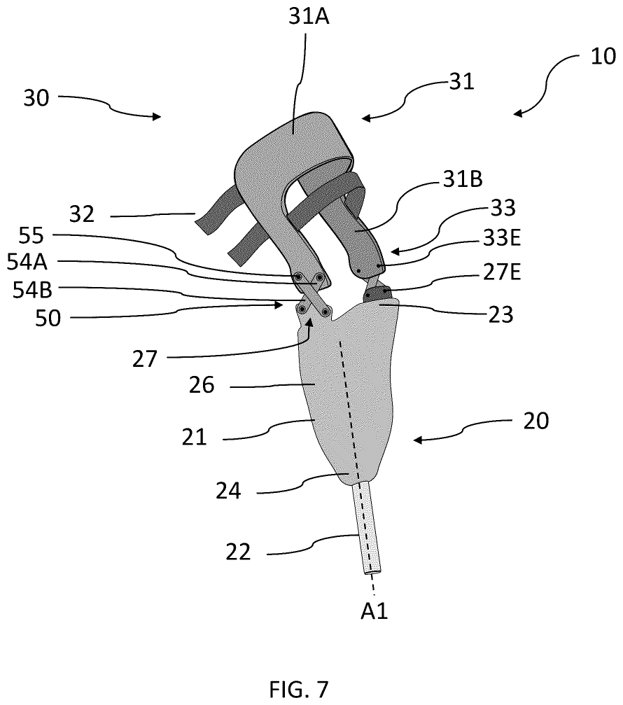

Referring now to FIG. 7, another embodiment of a hinge 40 for the assembly 10 is shown, in which an additional hinge element 50 is provided for securing the knee orthosis 30 to the socket 21. In this embodiment, the hinge 40 is a four-bar linkage-type mechanism. The femoral hinge element 33 includes hole 33E towards a lower extremity of each longitudinal member 31B. The socket hinge element 27 includes, towards the open end 23, two pairs of holes 27E on opposite sides of at an upper extremity of the open end 23. The additional hinge element 50 hingedly connects the knee orthosis 30 to the socket 21. In particular, the depicted additional hinge element 50 includes two sets of a first bar 54A and a second bar 54B, each bar 54A, 54B fastened at a first end thereof to a hole 33E in the longitudinal member 31B and at a second end thereof to a hole 27E in the socket 21, illustratively via fasteners 55. The bars 54A, 54B are arranged to allow relative motion between the knee orthosis 30 and the socket 21 for the required pivoting motion of the assembly 10. In shown embodiment, the bars 54A, 54B form an X or crossed shape. In other embodiments, other configurations of bars 54A, 54B could be contemplated.

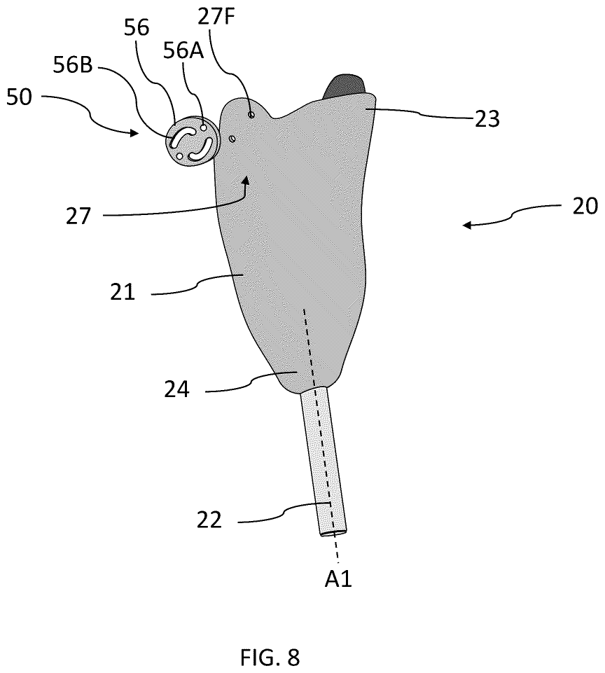

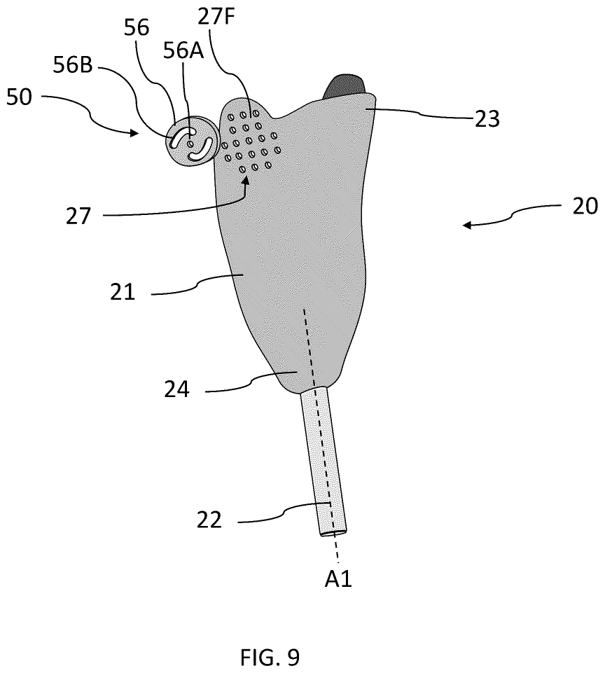

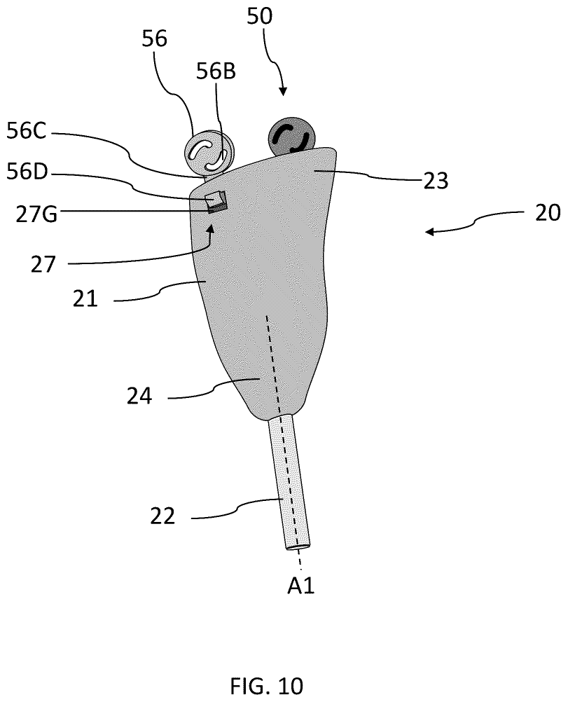

Referring now to FIGS. 8 and 9, another embodiment of a hinge 40 for the assembly is shown, in which an additional hinge element 50 is provided for securing the knee orthosis 30 (not shown) to the socket 21. In this embodiment, the socket hinge element 27 includes two sets of holes 27E on opposite sides of the socket 21 towards the open end 23. In FIG. 8, each set of holes 27E includes two holes 27E, while in FIG. 9, each set of holes 27E includes nineteen holes 27E. Other numbers of holes 27E are contemplated. The knee orthosis 30 also includes a set of holes towards the lower extremity of each longitudinal member 31B (not shown), the number of which may vary. The additional hinge element 50 includes a disk-like body 56 having holes 56A and guide slots 56B. In the embodiment shown in FIG. 8, each body 56 includes two holes 56A and two guide slots 56B, while in the embodiment shown in FIG. 9, each body 56 incudes one hole 56A and two guide slots 56B. Other numbers of holes 56A and guide slots 56B are contemplated. The holes 56A and guide slots 56B are each aligned with one or the other of the holes 27F in the socket 21 and the holes in the longitudinal member 31B of the knee orthosis 30. Fasteners (not shown), for instance pins or screws, are provided to secure the knee orthosis 30 to the socket 21, via the additional hinge element 50, in such a way to permit pivoting of the knee orthosis relative to the socket 21.

Referring to FIGS. 10-12, other embodiments of a hinge 40 for the assembly 10. In the following embodiments, the additional hinge element 50 is detachably secured to the socket hinge element 27 and rotatably or pivotally secured to the femoral hinge element 33 to allow the knee orthosis 30 to pivot relative to the socket 21. Stated differently, the additional hinge element 50 is solidly fixed to the socket hinge element 27, illustratively in a snap-fit type arrangement, but is detachable therefrom. As such, a patient can choose to use the socket 21 with or without the knee orthosis 30 with minimal adjustments required.

Referring to FIG. 10, another embodiment of a hinge 40 for the assembly 10 is shown, in which an additional hinge element 50 is provided for securing the knee orthosis 30 (not shown) to the socket 21. In this embodiment, the socket hinge element 27 includes a pair of cutouts 27G (or other like retaining features) on opposite sides of the socket 21 towards the open end 23. The cutouts 27G are configured for engaging the additional hinge elements 50, which also engage the lower extremity of the longitudinal member 31B of the knee orthosis 30, for instance via holes in the longitudinal member 31B (as described above). The additional hinge element 50 includes a body 56 having guide slots 56B, illustratively two guide slots 56B, for engaging the knee orthosis 30 in a pivoting manner, as described above. Each additional hinge element 50 further includes an extension 56C extending from the body 56 and a tab 56D at a distal end of the extension 56C. The tab 56D is inserted through a corresponding cutout 27G to secure the additional hinge element 50 to the socket 21. Other retention configurations are contemplated. As such, the additional hinge element 50 is fixedly secured to the socket 21 and rotatably or pivotally secured to the knee orthosis 30 (for instance via fasteners inserted through a respective slot 56B and a hole in the longitudinal member 31B) to allow the knee orthosis 30 to pivot relative to the socket 21.

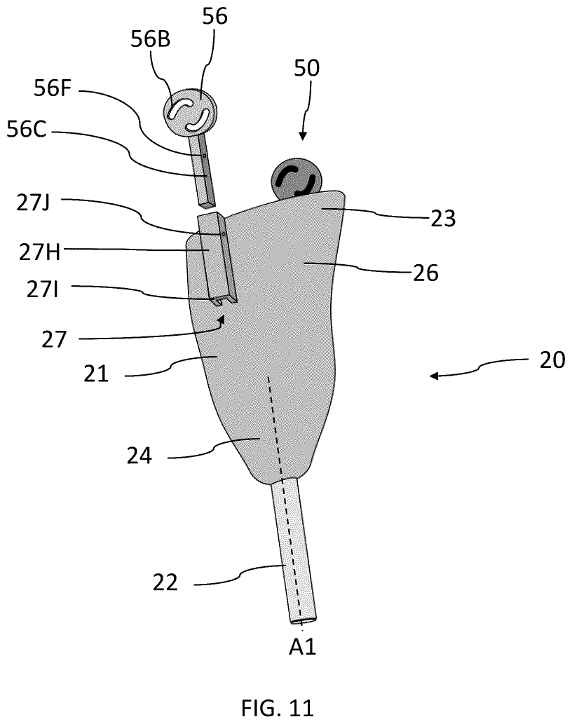

Referring to FIG. 11, another embodiment of a hinge 40 for the assembly is shown, in which an additional hinge element 50 is provided for securing the knee orthosis 30 (not shown) to the socket 21. Similarly to the embodiment shown in FIG. 10, the additional hinge element 50 acts as an intermediary between the socket 21 and the knee orthosis 30. In this embodiment, the socket hinge element 27 includes a pair of protrusions 27H protruding from of an outer surface 26 of opposite sides of the socket 21, towards the open end 23, with a slot 27I formed within each protrusion 27H. The slots 27I are configured for engaging the additional hinge elements 50, which also engage the lower extremity of the longitudinal member 31B of the knee orthosis 30, for instance via holes in the longitudinal member 31B (as described above). The additional hinge element 50 includes a body 56 having guide slots 56B, illustratively two guide slots 56B, for engaging the knee orthosis 30 in a pivoting manner, as described above. Each additional hinge element 50 further includes an extension 56C extending from the body 56. The extension 56C is inserted through a corresponding slot 27I in the protrusion 27H. One or more holes 27J in the protrusion 27H on the socket 21 is aligned with one or more holes 56F in the extension 56C of the hinge element 50, such that one or more fasteners (not shown) can be inserted through the holes 27J and 56F when aligned, to thereby secure the additional hinge element 50 to the socket 21. Other retention configurations are contemplated. As such, the additional hinge element 50 is fixedly secured to the socket 21 and rotatably or pivotally secured to the knee orthosis 30 (for instance via fasteners inserted through a respective slot 56B and a hole in the longitudinal member 31B) to allow the knee orthosis 30 to pivot relative to the socket 21.

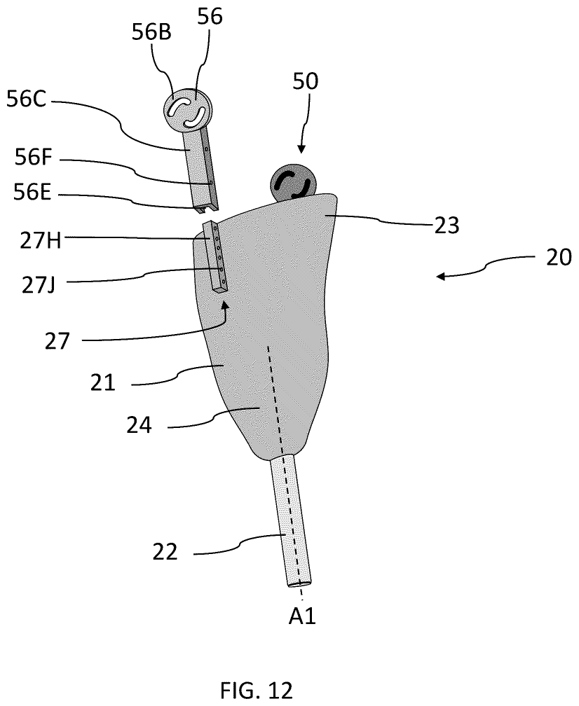

Referring to FIG. 12, another embodiment of a hinge 40 for the assembly is shown, in which an additional hinge element 50 is provided for securing the knee orthosis 30 (not shown) to the socket 21. Similarly to the embodiment shown in FIGS. 10 and 11, the additional hinge element 50 acts as an intermediary between the socket 21 and the knee orthosis 30. In this embodiment, the socket hinge element 27 includes a pair of protrusions 27H protruding from of an outer surface 26 of opposite sides of the socket 21, towards the open end 23, with holes 27J formed on one or more sides each protrusion 27H. The protrusions 27H and holes 27J are configured for engaging the additional hinge elements 50, which also engage the lower extremity of the longitudinal member 31B of the knee orthosis 30, for instance via holes in the longitudinal member 31B (as described above). The additional hinge element 50 includes a body 56 having guide slots 56B, illustratively two guide slots 56B, for engaging the knee orthosis 30 in a pivoting manner, as described above. Each additional hinge element 50 further includes an extension 56C extending from the body 56. The extensions 56C each include a slot 56E formed therewithin and holes 56F formed on one or more outer surfaces thereon. As such, the extensions 56C engage with the socket hinge elements 27 such that the protrusions 27H are received within the slots 56E, with the holes 27J, 56F aligned for securement therebetween (for instance, via fasteners). Other retention configurations are contemplated. As such, the additional hinge element 50 is fixedly secured to the socket 21 and rotatably or pivotally secured to the knee orthosis 30 (for instance via fasteners inserted through a respective slot 56B and a hole in the longitudinal member 31B) to allow the knee orthosis 30 to pivot relative to the socket 21.

Referring now to FIGS. 13A-13B, another aspect of the present disclosure, with like reference numerals referring to like elements. In this embodiment, the knee orthosis 30 is a unilateral brace. Stated differently, only one hinge 40 rotatably connects the femoral cuff 31 to the socket 21, on one of the medial side 30A or the lateral side 30B (illustratively on the lateral side 30B). The hinge 40 in this embodiment resembles the hinge 40 shown in FIG. 4, in which the femoral hinge element 33 includes a pair of holes 33A extending therethrough and the socket hinge element 27 (not shown) includes a pair of guide slots 27A (not shown) disposed therethrough. The holes 33A and guide slots 27A are adapted to be aligned and receive pins (not shown) therein, the pins rotatably fastening the socket 21 to the femoral cuff 31. It is understood, however, that other hinge 40 configurations are contemplated for the unilateral brace embodiment.

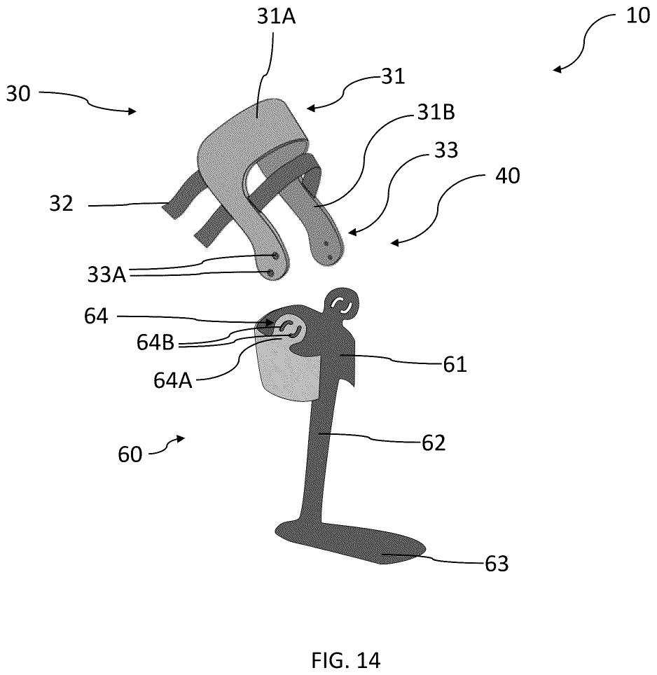

Referring now to FIG. 14, in another aspect of the present disclosure, a knee orthosis assembly 10 includes a knee orthosis 30 and an ankle foot orthosis 60, hereinafter referred to as AFO 60. The depicted knee orthosis 30 resembles the orthosis 30 shown in FIGS. 4 and 5, with like reference numerals referring to like elements. It is understood, however, that other knee orthoses disclosed herein and supporting the upper leg portion are similarly usable in the assembly 10 shown in FIG. 14. The AFO 60 is an external biomedical supportive device used for lower limbs to stabilize joints and is joinable to the knee orthosis 30. The depicted AFO 60 includes a shank cuff 61 receiving or engaging a lower leg portion of the patient. In the shown case, the shank cuff 61 has a curved shape configured to mate with a lower leg of the patient. The AFO 60 further includes a shank 62 coupled to the shank cuff 61 and engaging an ankle of the patient, and a foot plate 63 coupled to the shank 62 and supporting the patient's foot. Other AFO configurations are contemplated. In addition, AFO hinge elements 64 are provided at an upper extremity of the shank cuff 61 and is securable to corresponding femoral hinge elements 33 of a knee orthosis 30, for instance in the manners described above with reference to the various sockets 21. In the shown case, each AFO hinge element 64 includes a body 64A extending upwardly from an upper extremity of the shank cuff 61, and slots 64B through the body 64A. The slots are engageable with corresponding elements in the femoral hinge element 33 (i.e., via holes 33A), as discussed above, to pivotally connect the knee orthosis 30 to the AFO 60. Other AGO hinge elements 64 are contemplated.

The present disclosure provides an exemplary method of manufacturing a socket 21 for a leg prosthesis of a trans-tibial amputee, the socket engageable with a knee orthosis 30. A leg of the amputee is scanned to obtain information pertaining to the amputee's anatomy, in particular their stump. The socket 21 is modelled, for instance via 3D modelling, based on the amputee's anatomy, including modelling a socket hinge element 27 integrated to the socket 21 and engageable with a corresponding hinge element 33 of the knee orthosis 30. The socket 21 is then 3D printed with the socket hinge element 27 integrally formed therewith. It is understood that other processes beyond 3D printing may be contemplated to produce the socket 21. In addition, the above-described process for producing the socket 21 may also be used to produce the AFO 60.

It is noted that various connections are set forth between elements in the preceding description and in the drawings. It is noted that these connections are general and, unless specified otherwise, may be direct or indirect and that this specification is not intended to be limiting in this respect. A coupling between two or more entities may refer to a direct connection or an indirect connection. An indirect connection may incorporate one or more intervening entities. The term “connected” or “coupled to” may therefore include both direct coupling (in which two elements that are coupled to each other contact each other) and indirect coupling (in which at least one additional element is located between the two elements).

It is further noted that various method or process steps for embodiments of the present disclosure are described in the preceding description and drawings. The description may present the method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Furthermore, no element, component, or method step in the present disclosure is intended to be dedicated to the public regardless of whether the element, component, or method step is explicitly recited in the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various aspects of the present disclosure have been disclosed, it will be apparent to those of ordinary skill in the art that many more embodiments and implementations are possible within the scope of the present disclosure. For example, the present disclosure as described herein includes several aspects and embodiments that include particular features. Although these particular features may be described individually, it is within the scope of the present disclosure that some or all of these features may be combined with any one of the aspects and remain within the scope of the present disclosure. References to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. The use of the indefinite article “a” as used herein with reference to a particular element is intended to encompass “one or more” such elements, and similarly the use of the definite article “the” in reference to a particular element is not intended to exclude the possibility that multiple of such elements may be present.

The embodiments described in this document provide non-limiting examples of possible implementations of the present technology. Upon review of the present disclosure, a person of ordinary skill in the art will recognize that changes may be made to the embodiments described herein without departing from the scope of the present technology. Yet further modifications could be implemented by a person of ordinary skill in the art in view of the present disclosure, which modifications would be within the scope of the present technology.

What is claimed is:

1. A knee orthosis assembly for a trans-tibial amputee having a leg prosthesis, comprising:
a socket for receiving a stump of the trans-tibial amputee, the socket having an open end receiving the stump, the open end including a socket hinge element integrated with the open end; and
a femoral cuff configured for engaging an upper leg portion of the trans-tibial amputee, the femoral cuff having a femoral transverse member and a femoral longitudinal member protruding from the femoral transverse member, the femoral longitudinal member including a femoral hinge element integrated with the femoral longitudinal member, the femoral hinge element engaging with the socket hinge element to define a hinge of the knee orthosis assembly.

2. The knee orthosis assembly as defined in claim 1, wherein the socket hinge element directly engages the femoral hinge element.

3. The knee orthosis assembly as defined in claim 2, wherein the socket hinge element includes a first aperture and the femoral hinge element includes a second aperture aligned with the first aperture and rotatably secured to one another by a fastener inserted through the first and second apertures.

4. The knee orthosis assembly as defined in claim 2, wherein one of the socket hinge element and the femoral hinge element includes slots defined therethrough, and the other of the socket hinge element and the femoral hinge element includes apertures defined therethrough, the slots and apertures aligned and rotatably secured to one another by fasteners inserted therethrough.

5. The knee orthosis assembly as defined in claim 1, further comprising an additional hinge element, the additional hinge element interfacing the socket hinge element to the femoral hinge element.

6. The knee orthosis assembly as defined in claim 5, wherein the socket hinge element and the femoral hinge element each include holes and toothed portions, the toothed portions engaging one another to allow the femoral cuff to pivot with respect to the socket, and wherein the additional hinge element includes support arms engaging the holes in the socket hinge element and the femoral hinge element to secure the socket to the femoral cuff.

7. The knee orthosis assembly as defined in claim 5, wherein the socket hinge element and the femoral hinge element each include holes and the additional hinge element includes 4-bars links engaging the holes in the socket hinge element and the femoral hinge element to pivotally secure the socket to the femoral cuff.

8. The knee orthosis assembly as defined in claim 5, wherein the socket hinge element and the femoral hinge element each include holes and the additional hinge element includes a disk-shaped body having slots and holes disposed therethrough, the slots in the disk-shaped body engaging the holes in one of the socket hinge element and the femoral hinge element, the holes in the disk-shaped body engaging the holes in the other of the socket hinge element and the femoral hinge element to pivotally secure the socket to the knee orthosis assembly.

9. The knee orthosis assembly as defined in claim 5, wherein the additional hinge element is detachably secured to the socket hinge element and rotatably secured to the femoral hinge element.

10. The knee orthosis assembly as defined in claim 1, wherein an inner surface of the femoral cuff engages an outer surface of the socket adjacent the hinge.

11. The knee orthosis assembly as defined in claim 1, wherein an outer surface of femoral cuff engages an inner surface of the socket adjacent the hinge.

12. The knee orthosis assembly as defined in claim 1, wherein the socket hinge element includes a first aperture and the femoral hinge element includes a second aperture aligned with the first aperture and rotatably secured to one another by a fastener inserted through the first and second apertures.

13. The knee orthosis assembly as defined in claim 1, wherein one of the socket hinge element and the femoral hinge element includes slots defined therethrough and the other of the socket hinge element and the femoral hinge element includes apertures defined therethrough, the slots and apertures aligned and rotatably secured to one another by fasteners inserted therethrough.

14. The knee orthosis assembly as defined in claim 1, wherein the socket hinge element and the femoral hinge element each include holes and toothed portions, the toothed portions engaging one another to allow the femoral cuff to pivot with respect to the socket.

15. The knee orthosis assembly as defined in claim 1, further comprising 4-bar links engaging holes in the socket hinge element and the femoral hinge element to pivotally secure the socket to the femoral cuff.

16. The knee orthosis assembly as defined in claim 1, further comprising a disk-shaped body having slots and holes disposed therethrough, the slots in the disk-shaped body engaging holes in one of the socket hinge element and the femoral hinge element, the holes in the disk-shaped body engaging holes in the other of the socket hinge element and the femoral hinge element to pivotally secure the socket to the femoral cuff.

17. The knee orthosis assembly as defined in claim 1, further comprising an additional hinge element detachably secured to the socket hinge element and rotatably secured to the femoral hinge element.

18. A method of manufacturing a socket for a leg prosthesis of a trans-tibial amputee, the socket engageable with a knee orthosis, comprising:
scanning a leg of the trans-tibial amputee to obtain information pertaining to anatomy of the leg;
modelling the socket based on the anatomy of the leg, including modelling a socket hinge element integrated to the socket and engageable with a corresponding hinge element of the knee orthosis; and
3D printing the socket with the socket hinge element integrally formed therewith.

19. The method as defined in claim 18, wherein the modeling of the socket includes modeling the socket hinge element to be directly engageable with corresponding hinge element of the knee orthosis.

20. A knee orthosis assembly for a patient, comprising:
a femoral cuff configured for engaging an upper leg portion of the patient, the femoral cuff having a femoral transverse member and a femoral longitudinal member protruding from the femoral transverse member, the femoral longitudinal member including a femoral hinge element integrated with the femoral longitudinal member; and
an ankle-foot orthosis having a shank cuff receiving a lower leg portion of the patient, the shank cuff including an ankle-foot orthosis hinge element configured for engaging with the femoral hinge element, a shank coupled to the shank cuff and engaging an ankle of the patient, and a foot plate coupled to the shank and supporting a foot of the patient.
